(19) United States
(12) Patent Application Publication
(10) Pub. No.: US 2025/0346970 A1
(43) Pub. Date: Nov. 13, 2025

(54) METHODS FOR PROCESSING ENTRAINED SLAG INCLUSIONS IN STEEL WITH DEOXIDIZED CALCIUM WITH FIXED ALUMINUM

(71) Applicants: NORTH CHINA UNIVERSITY OF TECHNOLOGY, Beijing (CN); UNIVERSITY OF SCIENCE AND TECHNOLOGY BEIJING, Beijing (CN)

(72) Inventors: Lifeng ZHANG, Beijing (CN); Jujin WANG, Beijing (CN); Weijian WANG, Beijing (CN); Ying REN, Beijing (CN); Guojun CHEN, Beijing (CN); Bin GUO, Beijing (CN)

(73) Assignees: NORTH CHINA UNIVERSITY OF TECHNOLOGY, Beijing (CN); UNIVERSITY OF SCIENCE AND TECHNOLOGY BEIJING, Beijing (CN)

(21) Appl. No.: 18/797,434

(22) Filed: Aug. 7, 2024

(30) Foreign Application Priority Data
May 10, 2024 (CN) 202410573071.4

(51) Int. Cl. C21C 7/00

(52) U.S. Cl. CPC C21C 7/0006

(57) ABSTRACT

The embodiments of the present disclosure provide a method for processing entrained slag inclusions in steel with deoxidized calcium with fixed aluminum. The method proposes a kinetic model and further proposes a criterion for determining which composition of inclusions are the entrained slag inclusions based on the process of compositional transformation of the entrained slag inclusions. The method can clarify whether the inclusions in the steel are entrained slag or not, identify the source of the inclusions, and further provide a clear direction for the control of such inclusions in industrial production. Corresponding industrial measures can then be implemented to adjust steelmaking processes, control the occurrence of entrained slag inclusions, reduce the count of entrained slag inclusions in steel, and enhance process efficiency and steel product quality.

### CROSS-REFERENCE TO RELATED APPLICATIONS

This application claims priority to Chinese Patent Application 202410573071.4, filed on May 10, 2024, the entire content of which is incorporated herein by reference.

### TECHNICAL FIELD

The present disclosure relates to the field of metallurgical technology, and in particular relates to a method for processing entrained slag inclusions in steel with deoxidized calcium with fixed aluminum.

### BACKGROUND

Large-size inclusions in molten steel are mostly detrimental to the quality of the steel, and the presence of these inclusions may seriously damage the surface smoothness and mechanical properties of the produced steel. The sources of these large-size inclusions are varied, which may include the growth of deoxidation products, entrainment of refined slag, spalling of refractory materials, detachment of nozzle clogging, and entrainment of tundish covering agents and mold fluxes. Determining the exact source of the large-size inclusions is a problem to be solved. Tracer tests are one of the widely used techniques for analyzing the sources of the large-size inclusions. The tracer tests involve the use of a variety of substances as tracers, which may help to determine the source of the large-size inclusions. The tracers may include rare earth oxides, CoO, Pr6O11, Na2O, K2O, BaO, or the like. These tracers are added to the refined slag during the production process, and their presence and distribution in the inclusions may provide valuable information for understanding the source of the inclusions. By conducting tracer tests with different tracers, researchers may study changes in the composition of the inclusions and determine their sources, which may be used to develop strategies to minimize these harmful inclusions and improve the overall quality of the steel.

While tracer tests helped to determine the sources of the large-size inclusions, there is still a lack of precise criteria to determine whether the inclusions originate from slag entrainment. The reason for this problem is that the entrained slag continues to react with the molten steel after it has been mixed into the molten steel, resulting in a change in the composition of the entrained slag. Additionally, a reaction occurs between the tracer-containing refined slag and the molten steel, which displaces tracer elements from the molten steel, and finally transfers the tracer elements from the refined slag to the molten steel, and ultimately to the inclusions.

Therefore, it is desirable to provide a method for processing entrained slag inclusions in steel with deoxidized calcium with fixed aluminum, in order to determine whether or not the inclusions in the molten steel are the entrained slag inclusions.

### SUMMARY

One of the embodiments of the present disclosure provides a method for processing entrained slag inclusions in steel with deoxidized calcium with fixed aluminum, wherein: an initial composition of inclusions is the same as a composition of a refined slag, including CaO, Al₂O₃, SiO₂, MnO, MgO, CaS, and an added tracer La₂O₃, and a reaction Equation (1) is shown as below;

$$x [ M ] + y [ O ] = ( {M}_{x} {O}_{y} ) \qquad ( 1 )$$

wherein M denotes any one of elements of calcium (Ca), aluminum (Al), silicon (Si), manganese (Mn), magnesium (Mg), or lanthanum (La), O denotes an element of oxygen (O) or an element of sulfur (S), and x and y are positive integers;
the inclusions are in a liquid state and exist in molten steel in a spherical shape, a size of the inclusions remains constant during a reaction process, and for a chemical reaction shown in the Equation (1), a change in Gibbs free energy is generated according to an Equation (2):

$$Δ G = Δ {G}^{⊖} + RTln \frac{{a}_{{M}_{x} {O}_{y}}^{*}}{{a}_{M}^{* x} \cdot {a}_{O}^{* y}} \qquad ( 2 )$$

wherein ΔG denotes the change in Gibbs free energy of the chemical reaction, unit in J/mol, ΔG^{Θ} denotes a standard change in Gibbs free energy, unit in J/mol, R denotes a gas constant, unit in J/(mol-K), T denotes a temperature of the chemical reaction, unit in K, a denotes an activity, and * represents an interface between the inclusions and the molten steel;
an activity of the component M is generated through Equations (3) and (4):

$${a}_{M} = {f}_{M} \cdot [ M ] \qquad ( 3 )$$

$${lgf}_{M} = {\sum }_{j} {e}_{M}^{j} [ j ] + {\sum }_{j} {\sum }_{k} {r}_{M}^{j , k} [ j ] [ k ] \qquad ( 4 )$$

wherein a_{M} denotes the activity of the component M, ƒ_{M} denotes an activity coefficient of the component M, [M] denotes a mass fraction of the component M in the molten steel, unit in %, e denotes a first-order activity interaction coefficient, r denotes a second-order activity interaction coefficient, and components of the molten steel include Fe—C—Si—Mn—P—S—Ca—Al—Mg—O, and [j] and [k] denote mass fractions of a j-th component and a k-th component of the molten steel respectively, unit in %, j and k are positive integers;
an activity of a molecular component MₓO_{y} is generated based on an ion and molecule coexistence theory (IMCT), through Equations (5) and (7), and an activity of an ionic component MₓO_{y} is generated through Equations (6) and (7):

$${a}_{{M}_{x} {O}_{y}} = \frac{{N}_{{M}_{x} {O}_{y}}}{{N}_{t}} \qquad ( 5 )$$

$${a}_{{M}_{x} {O}_{y}} = \frac{( x + y ) {N}_{{M}_{x} {O}_{y}}^{′}}{{N}_{t}} \qquad ( 6 )$$

$${N}_{t} = \sum {N}_{{M}_{x} {O}_{y}} + \sum ( x + y ) {N}_{{M}_{x} {O}_{y}}^{′} \qquad ( 7 )$$

wherein N_{M}_{ₓ}_{O}_{_{y}} denotes a molar amount of the molecular component MₓO_{y}, unit in mol, N_{M}_{ₓ}_{O}_{_{y}}′ denotes a molar amount of the ionic component MₓO_{y}, unit in mol, and Nₜ denotes a total molar amount of all components of the molten steel, including simple compounds and complex compounds generated by the simple compounds, unit in mol;
mass transfer flux equations for the component M in the molten steel and the component MₓO_{y} in the inclusions within the interfacial layer are:

$${J}_{M} = - \frac{{ρ}_{st} {k}_{M}}{100 {M}_{M}} ( {[ M ]}^{b} - [ M ] \text{*)} \qquad ( 8 )$$

$${J}_{( {M}_{x} {O}_{y} )} = - \frac{{ρ}_{inc} {k}_{{M}_{x} {O}_{y}}}{100 {M}_{{M}_{x} {O}_{y}}} ( {[ {M}_{x} {O}_{y} ]}^{b} - [ {M}_{x} {O}_{y} ] \text{*)} \qquad ( 9 )$$

wherein J_{M} denotes a mass transfer flux of the component M in the molten steel, unit in mol/(m2·s), J_{(M}_{ₓ}_{O}_{_{y}}₎ denotes a mass transfer flux of the component MₓO_{y} in the inclusions, unit in mol/(m2·s), ρₛₜ denotes a density of the molten steel, unit in kg/m3, ρ_{inc} denotes a density of the inclusions, unit in kg/m3, k_{M} denotes a mass transfer coefficient of the component M in the molten steel, unit in m/s, k_{M}_{ₓ}_{O}_{_{y}} denotes a mass transfer coefficient of the component MₓO_{y} in the inclusions, unit in m/s, M_{M} denotes a molecular weight of the component M in the molten steel, unit in kg/mol, M_{M}_{ₓ}_{O}_{_{y}} denotes a molecular weight of the component MₓO_{y} in the inclusions, unit in kg/mol, [MₓO_{y}] denotes a mass fraction of the component MₓO_{y} in the molten steel, unit in %, and b represents the molten steel body; a sum of the mass transfer flux of the component M in the steel and the mass transfer flux of the component MₓO_{y} in the inclusions is conserved, as shown in an Equation (10):

$${J}_{M} + {J}_{( {M}_{x} {O}_{y} )} = 0 \qquad ( 10 )$$

a total flux of cations and a total flux of anions are equal to maintain an electrically neutral environment, as shown in an Equation (11);

$${\sum }_{M} {n}_{M} {J}_{M} - 2 {J}_{o} - 2 {J}_{s} = 0 \qquad ( 11 )$$

wherein n_{M} denotes a charge number of M, J_{O} denotes a mass transfer flux of the element of O in the molten steel, unit in mol/(m2·s), and J_{S} denotes a mass transfer flux of the element of S in the molten steel, unit in mol/(m2·s);
a concentration of each component within the interface is obtained based on the Equation (2), the Equation (10), and the Equation (11);
a change in the composition of the inclusions over a time step is generated based on an Equation (12):

$$\frac{d ( [ {M}_{x} {O}_{y} ] )}{dt} = - \frac{{A}_{int} {k}_{{M}_{x} {O}_{y}}}{{V}_{inc}} ( [ {M}_{x} {O}_{y} ] * - {[ {M}_{x} {O}_{y} ]}^{b} ) \qquad ( 12 )$$

wherein Aᵢₙₜ denotes an area of the interface, unit in m², V_{inc} denotes a volume of the inclusions, unit in m³, k_{M}_{ₓ}_{O}_{_{y}} is 0.1 times of k_{M}, and k_{M} is determined based on Equations (13) and (14):

$${k}_{M} = 0.2 {( \frac{{D}_{M} \cdot {u}_{slip}}{π {d}_{inc}} )}^{1 / 2} \qquad ( 13 )$$

$${u}_{sinp} = \frac{( {ρ}_{m} - {ρ}_{inc} ) {d}_{inc}^{2} g}{18 {μ}_{st}} \qquad ( 14 )$$

wherein uₛₗᵢₚ denotes a relative diffusion velocity between the molten steel and the inclusions, unit in m/s, d_{inc} denotes the size of the inclusions, in m, g denotes an acceleration of gravity, unit in m/s², μₛₜ denotes a viscosity of the molten steel, unit in Pa·s, and D_{M} denotes a diffusion rate of the component M in the molten steel, unit in m/s.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a flowchart illustrating an exemplary process for processing entrained slag inclusions in steel with deoxidized calcium with fixed aluminum according to some embodiments of the present disclosure;

FIG. 2a is a schematic diagram illustrating evolution of a composition of entrained slag inclusions with an initial La₂O₃ content of 1%;

FIG. 2b is a schematic diagram illustrating evolution of a composition of entrained slag inclusions with an initial La₂O₃ content of 5%;

FIG. 2c is a schematic diagram illustrating evolution of a composition of entrained slag inclusions with an initial La₂O₃ content of 20%;

FIG. 2d is a schematic diagram illustrating evolution of a composition of entrained slag inclusions with an initial La₂O₃ content of 50%;

FIG. 3a is a schematic diagram illustrating a change in a composition of entrained slag inclusions over time with an initial La₂O₃ content of 5%;

FIG. 3b is a schematic diagram illustrating a change in a composition of entrained slag inclusions over time with an initial La₂O₃ content of 20%;

FIG. 3c is a schematic diagram illustrating a change in a composition of entrained slag inclusions over time with an initial La₂O₃ content of 50%;

FIG. 4a is a schematic diagram illustrating a compositional distribution of CaO in entrained slag inclusions with an initial La₂O₃ content of 5%;

FIG. 4b is a schematic diagram illustrating a compositional distribution of Al₂O₃ in entrained slag inclusions with an initial La₂O₃ content of 5%;

FIG. 4c is a schematic diagram illustrating a compositional distribution of MgO in entrained slag inclusions with an initial La₂O₃ content of 5%;

FIG. 4d is a schematic diagram illustrating a compositional distribution of La₂O₃ in entrained slag inclusions with an initial La₂O₃ content of 5%;

FIG. 5 is a schematic diagram illustrating a change in a proportion of entrained slag inclusions in steel processed with aluminum deoxidation and calcium treatment;

FIG. 6 is a flowchart illustrating an exemplary process of processing entrained slag inclusions in steel with deoxidized calcium with fixed aluminum according to some embodiments of the present disclosure.

### DETAILED DESCRIPTION

In order to more clearly illustrate the technical solutions of the embodiments of the present disclosure, the accompanying drawings to be used in the description of the embodiments will be briefly described below. Obviously, the accompanying drawings in the following description are only some examples or embodiments of the present disclosure, and that the present disclosure may be applied to other similar scenarios in accordance with these drawings without creative labor for those of ordinary skill in the art. Unless obviously acquired from the context or the context illustrates otherwise, the same numeral in the drawings refers to the same structure or operation.

It should be understood that “system,” “device,” “unit,” and/or “module” as used herein is a way to distinguish between different components, elements, parts, sections, or assemblies at different levels. However, these words may be replaced by other expressions if they accomplish the same purpose.

As indicated in the present disclosure and in the claims, the singular forms “a,” “an,” and “the” may be intended to include the plural forms as well, unless the context clearly indicates otherwise. In general, the terms “comprise,” “comprises,” and/or “comprising,” “include,” “includes,” and/or “including,” when used in this disclosure, specify the presence of stated features, integers, steps, operations, elements, and/or components, but do not preclude the presence or addition of one or more other features, integers, steps, operations, elements, components, and/or groups thereof.

Flowcharts are used in the present disclosure to illustrate the operations performed by the system according to some embodiments of the present disclosure. It should be understood that the operations described herein are not necessarily executed in a specific order. Instead, the operations may be executed in reverse order or simultaneously. Additionally, one or more other operations may be added to these processes, or one or more operations may be removed from these processes.

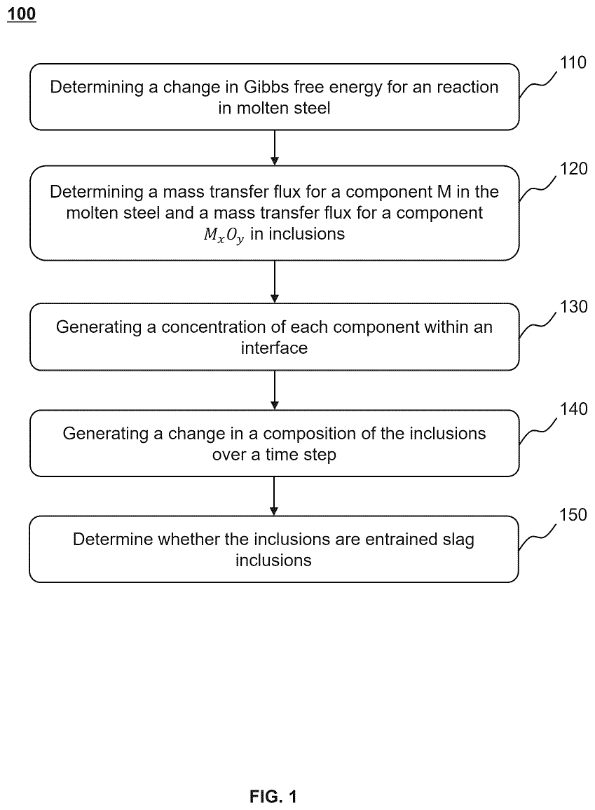

FIG. 1 is a flowchart illustrating an exemplary process of a method for processing entrained slag inclusions in steel with deoxidized calcium with fixed aluminum according to some embodiments of the present disclosure.

As shown in FIG. 1, a process 100 may include the following operations. In some embodiments, the process 100 may be executed by an analysis processor of an analytical terminal.

The analytical terminal refers to a terminal device for determining whether an inclusion is an entrained slag inclusion. In some embodiments, the analytical terminal may include an analysis processor and an analytical data storage. In some embodiments, the analytical terminal may be a computer, a laptop, a server, etc.

The analysis processor may be configured to process data related to the method for processing entrained slag inclusions in steel with deoxidized calcium with fixed aluminum. In some embodiments, the analysis processor may be a central processing unit (CPU), a programmable logic controller (PLC), or the like. In some embodiments, the analysis processor may be a single server or a server group. The server group may be centralized or distributed. In some embodiments, the analysis processor may be local or remote. In some embodiments, the analysis processor may be implemented on a cloud platform. By way of example only, the cloud platform may include a private cloud, a public cloud, a hybrid cloud, a community cloud, a distributed cloud, an on-premises cloud, a multi-tiered cloud, or any combination thereof.

The analytical data storage may store data, instructions, and/or any other

information. In some embodiments, the analytical data storage may store data and/or instructions related to the method for processing entrained slag inclusions in steel with deoxidized calcium with fixed aluminum. In some embodiments, the analytical data storage may be a hard drive, a USB flash drive, etc.

In 110, determining a change in Gibbs free energy for a reaction in molten steel.

The molten steel is a steelmaking material in a molten state. In some embodiments, the molten steel may be obtained by heating at a high temperature.

An inclusion is a foreign substance that is mixed into the molten steel during the steel smelting process. For example, the inclusions may include entrained slag inclusions, crystallizer protection slag inclusions, refractory material inclusions, or the like.

The Gibbs free energy is a thermodynamic state function defined by certain thermodynamic derivations at constant temperature and pressure.

Since an initial composition of inclusions is the same as a composition of a refined slag, including CaO, Al₂O₃, SiO₂, MnO, MgO, CaS, and an added tracer La₂O₃, a reaction Equation (1) is shown as below;

$$x [ M ] + y [ O ] = ( {M}_{x} {O}_{y} ) \qquad ( 1 )$$

wherein M denotes any one of elements of calcium (Ca), aluminum (Al), silicon (Si), manganese (Mn), magnesium (Mg), or lanthanum (La), O denotes an element of oxygen (O) or an element of sulfur (S), and x and y are positive integers.

All chemical reactions considered in a kinetic model are shown in Table 1. Since the inclusions are in a liquid state and exist in molten steel in a spherical shape, a size of the inclusions remains constant during the reactions. Additionally, since the total amount of entrained slag inclusions is small compared to the molten steel, changes in the composition of the molten steel due to these reactions are neglected.

TABLE 1
Chemical reactions considered in the kinetic model of a relationship
between the entrained slag inclusions and the molten steel

| Chemical reaction | Standard Gibbs energy change (J/mol) |
|---|---|
| [Ca] + [O] = (CaO) | ΔG^{Θ} = −138240.86 − 63.0T |
| 2[Al] + 3[O] = (Al₂O₃) | ΔG^{Θ} = −1225000 + 393.8T |
| [Si] + 2[O] = (SiO₂) | ΔG^{Θ} = −581900 + 221.8T |
| [Mg] + [O] = (MgO) | ΔG^{Θ} = −89960 − 82.0T |
| [Mn] + [O] = (MnO) | ΔG^{Θ} = 288150 − 1283T |
| 2[La] + 3[O] = (La₂O₃) | ΔG^{Θ} = −1443880 + 337T |
| [Ca] + [S] = (CaS) | ΔG^{Θ} = −542531 + 124.15T |

In some embodiments, for each reaction shown in Equation (1), the analytical processor may generate a change in Gibbs free energy, according to Equation (2):

$$Δ G = Δ {G}^{⊖} + RTln \frac{{a}_{{M}_{x} {O}_{y}}^{*}}{{a}_{M}^{* x} \cdot {a}_{O}^{* y}} \qquad ( 2 )$$

wherein ΔG denotes the change in Gibbs free energy of the chemical reaction, unit in J/mol, ΔG^{Θ} denotes a standard change in Gibbs free energy, unit in J/mol, R denotes a gas constant, unit in J/(mol-K), T denotes a temperature of the chemical reaction, unit in K, a denotes an activity, and * represents an interface between the inclusions and the molten steel.

The activity refers to an effective concentration or effective mole fraction of different components of the molten steel and the inclusions involved in the reaction.

In some embodiments, the analytical processor may retrieve data from Table 2 in the analytical data storage to generate activity interaction coefficients. Table 2 includes the activity interaction coefficients corresponding to different elements. Table 2 may be determined manually based on prior experience and entered into the analytical terminal. The activity of [La] in the steel is simplified and assumed to be 1.0 due to the lack of relevant parameters. The activity interaction coefficients between other elements are shown in Table 2. In some embodiments, the analytical processor may generate the activity of each component of the molten steel, using the Wagner model, via Equation (3) and Equation (4):

$${a}_{M} = {f}_{M} \cdot [ M ] \qquad ( 3 )$$

$${lgf}_{M} = {\sum }_{j} {e}_{M}^{j} [ j ] + {\sum }_{j} {\sum }_{k} {r}_{M}^{j , k} [ j ] [ k ] \qquad ( 4 )$$

wherein a_{M} denotes the activity of the component M, ƒ_{M} denotes an activity coefficient of the component M, [M] denotes a mass fraction of the component M in the molten steel, unit in %, e denotes a first-order activity interaction coefficient, r denotes a second-order activity interaction coefficient, and components of the molten steel include Fe—C—Si—Mn—P—S—Ca—Al—Mg—O, and [j] and [k] denote mass fractions of a j-th component and a k-th component of the molten steel respectively, unit in %, j and k are positive integers.

TABLE 2
Activity interaction coefficients between components in the molten steel (1% standard state)

| | e_{M}^{j}, r_{M}^{j} | | | | | | | |
|---|---|---|---|---|---|---|---|---|
| M | Al | C | Ca | Mg | Mn | O | S | Si |
| Al | 80.5/T | 0.091 | −0.047 | −0.019 | 0 | −9720/T + 3.21 | 0.035 | 0.056 |
| | (0.17/ | (−0.004) | | | | (2.8e5/T − 107) | | (−0.0006) |
| | T − 0.0011) | | | | | [−13.78/T − 0.021] | | |
| C | 0.043 | 158/ | −0.097 | 0.07 | −299.5/ | −0.34 | 0.044 | 162/ |
| | (−0.0007) | T + 0.0581 | | | T + 0.15423 | | | T − 0.008 |
| | | (8.94/ | | | | | | (1.94/ |
| | | T + 0.0026) | | | | | | T − 0.0003) |
| Ca | −0.072 | −0.335 | −0.002 | 0 | −0.0156 | −2500 | −140 | −0.096 |
| | (0.0007) | (0.012) | | | | (2.6e5) [2.1e5] | | (0.0009) |
| Mg | −0.017 | 0.15 | 0 | 0 | 0 | −2.6e6/T + 958 | 0 | −0.09 |
| | | | | | | (4.2e9/T − 1.9e6) | | |
| | | | | | | [−5.2e8/T + 2.1e5] | | |
| Mn | 0 | −1370/ | −0.023 | 0 | 0 | −0.083 | −0.048 | −0.0327 |
| | | T + 0.690 | | | | | | |
| O | −5760/ | −0.45 | −990 | −1.7e6/ | −0.021 | −1750/ | −0.133 | −0.066 |
| | T + 1.90 | | (4.2e4) | T + 630 | | T + 0.76 | | |
| | (−25.0/ | | [2.1e5] | (−1.7e8/ | | | | |
| | T + 0.0033) | | | T + 70500) | | | | |
| | [3.3e5/ | | | [5.6e9/ | | | | |
| | T + 127.3] | | | T − 2.5e6] | | | | |
| S | 0.041 | 0.111 | −110 | 0 | −0.026 | −0.27 | −120/ | 0.075 |
| | | (0.0058) | | | | | T + 0.018 | |
| Si | 0.058 | 378/ | −0.066 | −0.105 | −0.0146 | −0.119 | 0.066 | 0.103 |
| | | T − 0.0245 | | | | | | |

Notes:
( ) denotes rᵢ^{j}; [ ] denotes rᵢ^{ji}; T denotes temperature, in K.

The molten steel and the inclusions include molecular components and ionic components. For example, CaS is an ionic component and La₂O₃ is a molecular component. In some embodiments, the analytical processor may generate an activity of a molecular component MₓO_{y} via Equation (5) and Equation (7) and an activity of an ionic component MₓO_{y} via Equation (6) and Equation (7) based on an ion and molecule coexistence theory (IMCT):

$${a}_{{M}_{x} {O}_{y}} = \frac{{N}_{{M}_{x} {O}_{y}}}{{N}_{t}} \qquad ( 5 )$$

$${a}_{{M}_{x} {O}_{y}} = \frac{( x + y ) {N}_{{M}_{x} {O}_{y}}^{′}}{{N}_{t}} \qquad ( 6 )$$

$${N}_{t} = \sum {N}_{{M}_{x} {O}_{y}} + \sum ( x + y ) {N}_{{M}_{x} {O}_{y}}^{′} \qquad ( 7 )$$

wherein N_{M}_{ₓ}_{O}_{_{y}} denotes a molar amount of the molecular component MₓO_{y}, unit in mol, N_{M}_{ₓ}_{O}_{_{y}}′ denotes a molar amount of the ionic component MₓO_{y}, unit in mol, and Nₜ denotes a total molar amount of all components of the molten steel, including simple compounds and complex compounds generated by the simple compounds, unit in mol.

In 120, determining a mass transfer flux of the component M in the molten steel and a mass transfer flux of the component MₓO_{y} in the inclusions.

The interface refers to a reaction surface where the molten steel and the inclusions react. In some embodiments, the interface may be a surface of the inclusions in the molten steel.

The mass transfer flux refers to an amount of components in the molten steel/the inclusions passing through unit area perpendicular to a direction of mass transfer in unit time.

According to the two-film theory, a boundary layer exists on two sides of the interface between the inclusions and the steel, and reactions occur only in the boundary layer. Reactants and reaction products diffuse within the interface. Components in both the inclusion and the molten steel are uniformly distributed, with concentration gradients existing only within the boundary layer. At elevated reaction temperatures, chemical reactions at the interface do not control the reaction rate; instead, species diffusion within the boundary layer controls the reaction rate. Chemical reactions at the interface always maintain equilibrium, and the concentration gradient of components from the interface to the bulk is the driving force for diffusion.

In some embodiments, the analytical processor may generate mass transfer fluxes of different steel or inclusion components based on mass transfer flux equations. For example, the mass transfer flux equations may be represented by Equation (8) and Equation (9):

$${J}_{M} = - \frac{{ρ}_{st} {k}_{M}}{100 {M}_{M}} ( {[ M ]}^{b} - [ M ] \text{*)} \qquad ( 8 )$$

$${J}_{( {M}_{x} {O}_{y} )} = - \frac{{ρ}_{inc} {k}_{{M}_{x} {O}_{y}}}{100 {M}_{{M}_{x} {O}_{y}}} ( {[ {M}_{x} {O}_{y} ]}^{b} - [ {M}_{x} {O}_{y} ] \text{*)} \qquad ( 9 )$$

wherein J_{M} denotes a mass transfer flux of the component M in the molten steel, unit in mol/(m²·s), J_{(M}_{ₓ}_{O}_{_{y}}₎ denotes a mass transfer flux of the component MₓO_{y} in the inclusions, unit in mol/(m²·s), ρₛₜ denotes a density of the molten steel, unit in kg/m3, ρ_{inc} denotes a density of the inclusions, unit in kg/m3, k_{M} denotes a mass transfer coefficient of the component M in the molten steel, unit in m/s, k_{M}_{ₓ}_{O}_{_{y}} denotes a mass transfer coefficient of the component MₓO_{y} in the inclusions, unit in m/s, M_{M} denotes a molecular weight of the component M in the molten steel, unit in kg/mol, M_{M}_{ₓ}_{O}_{_{y}} denotes a molecular weight of the component MₓO_{y} in the inclusions, unit in kg/mol, [MₓO_{y}] denotes a mass fraction of the component MₓO_{y} in the molten steel, unit in %, and b represents the molten steel body.

According to the principle of conservation of mass, there is no accumulation of matter within the interface. Therefore, a sum of the mass transfer flux of the component MₓO_{y} in the molten steel and the mass transfer flux of the component MₓO_{y} in the inclusions is conserved, as shown in Equation (10):

$${J}_{M} + {J}_{( {M}_{x} {O}_{y} )} = 0 \qquad ( 10 )$$

A total flux of cations and a total flux of anions are equal to maintain an electrically neutral environment, as shown in Equation (11);

$${\sum }_{M} {n}_{M} {J}_{M} - 2 {J}_{o} - 2 {J}_{s} = 0 \qquad ( 11 )$$

wherein n_{M} denotes a charge number of M, J_{O} denotes a mass transfer flux of the element of O in the molten steel, unit in mol/(m²·s), and J_{S} denotes a mass transfer flux of the element of S in the molten steel, unit in mol/(m²·s).

In 130, generating a concentration of each component within the interface.

Since the interface is in equilibrium at all times, ΔG=0, i.e., Equation (2) equals zero. In some embodiments, the analytical processor may generate, by calculation, concentrations of different molten steel components or composition of the inclusions within the interface, based on Equation (2), Equation (10), and Equation (11).

In 140, generating a change in the composition of the inclusions over a time step.

The time step refers to a difference between two consecutive time points. In some embodiments, the time step may be the difference between time points during a reaction process of molten steel and inclusions. For example, the time step may be 1 second. The time step may be pre-set based on experience.

The change in the composition of the inclusions refers to a change in the content of different components in the inclusions. For example, the change in the composition of the inclusions may include a change in molar concentration or a change in mass fraction, etc.

In some embodiments, provided that concentrations of different molten steel components or concentrations of different inclusion components within the interface are known, the analytical processor may generate the change in the composition of the inclusions over a time step using Equation (12):

$$\frac{d ( [ {M}_{x} {O}_{y} ] )}{dt} = - \frac{{A}_{int} {k}_{{M}_{x} {O}_{y}}}{{V}_{inc}} ( [ {M}_{x} {O}_{y} ] * - {[ {M}_{x} {O}_{y} ]}^{b} ) \qquad ( 12 )$$

wherein Aᵢₙₜ denotes an area of the interface, in m², V_{inc} denotes a volume of the inclusions, in m³, MₓO_{y} denotes the mass fraction of the MₓO_{y} component in the inclusions, with k_{M}_{ₓ}_{O}_{_{y}} being 0.1 times of k_{M}.

In some embodiments, the analytical processor may determine the k_{M} by Equation (13) and Equation (14):

$${k}_{M} = 0.2 {( \frac{{D}_{M} \cdot {u}_{slip}}{π {d}_{inc}} )}^{1 / 2} \qquad ( 13 )$$

$${u}_{sinp} = \frac{( {ρ}_{m} - {ρ}_{inc} ) {d}_{inc}^{2} g}{18 {μ}_{st}} \qquad ( 14 )$$

wherein uₛₗᵢₚ denotes a relative diffusion velocity between the molten steel and the inclusions, unit in m/s, d_{inc} denotes the size of the inclusions, in m, g denotes an acceleration of gravity, unit in m/s², μₛₜ denotes a viscosity of the molten steel, unit in Pa·s, and D_{M} denotes a diffusion rate of the component M in the molten steel, unit in m/s.

In some embodiments, the analytical processor may generate, through a composition model, the change in the composition of the inclusions based on a reaction time, an initial composition, a volume of the inclusions, the mass transfer coefficient of the component M in the molten steel, and an average size of the inclusions.

In some embodiments, the composition model may be a deep learning neural network model. Exemplary deep learning neural network models may include a neural network model (NN) model, a deep neural network (DNN) model, a recurrent neural network (RNN) model, or the like, or or any combination thereof.

In some embodiments, an input of the composition model may include the reaction time, the initial composition, the volume of the inclusions, the mass transfer coefficient of the component M in the molten steel, and the average size of the inclusions, and an out put of the composition model may include a predicted change in the composition of the inclusions.

The reaction time refers to a time during which the molten steel and the inclusions react. For example, the reaction time may be a time in a range of 0 s to 10000 s.

The initial composition refers to compositional components of the inclusions in the molten steel prior to reaction. In some embodiments, the initial composition may be mass fractions of the compositional components of the inclusions. For example, the initial composition may be (0 wt %, 20 wt %, 15 wt %, 32 wt %, 5 wt %), which may indicate that the initial compositions of CaO, Al₂O₃, MgO, and La₂O₃ in the inclusions are 20 wt %, 15 wt %, 32 wt %, and 5 wt %, respectively.

The volume of the inclusions refers to an average volume of the inclusions in the molten steel. For example, the volume of the inclusions may be 1 μm³, 750 μm³, etc. In some embodiments, the volume of the inclusions may be determined based on an average size of the inclusions.

The analytical processor may determine the mass transfer coefficient (k_{M}) of the component M in the molten steel based on equation (13). For example, the mass transfer coefficient of the component M in the molten steel may be expressed as (K, 0.18, 0.21, 0.14), indicating that the mass transfer coefficients of the components [Ca], [Al], and [Si] in the molten steel are 0.18, 0.21, and 0.14, respectively.

The average size of the inclusions refers to an average size of the plurality of inclusions in the molten steel. For example, the average size of the inclusions may be an average diameter or an average volume of the inclusions. Because the smaller the average size of the inclusions is, the larger the area of the interface, and the faster the rate of reaction when the volume of the inclusions is fixed, it is necessary to take the average size of the inclusions into account when predicting the change in the composition of the inclusions.

The changes in the composition of the inclusions refers to a rate of change in the composition of the inclusions in the molten steel over at least one time step. For example, the change in the composition of the inclusions may be expressed as [(Qt1, 0.4, 0.03, 0.24, 0.06), (Qt2, 0.3, 0.05, 0.26, 0.05), (Qt3, 0.4, 0.04, 0.22, 0.07) . . . ], which inidicate that the change in the composition of CaO, Al₂O₃, MgO, and La₂O₃ in the inclusions over a time step t1 are 0.4 wt %/s, 0.03 wt %/s, 0.24 wt %/s, and 0.06 wt %/s, respectively; the change in the composition of CaO, Al₂O₃, MgO, and La₂O₃ in the inclusions over a time step t2 are 0.3 wt %/s, 0.05 wt %/s, 0.26 wt %/s, and 0.05 wt %/s, respectively; and the change in the composition of CaO, Al₂O₃, MgO, and La₂O₃ in the inclusions over a time step t3 are 0.4 wt %/s, 0.04 wt %/s, 0.22 wt %/s, 0.07 wt %/s, respectively.

Understandably, since different reaction times correspond to different reaction rates, the rate of change in the composition of the inclusions varies under different reaction rates. As a result, changes in the composition of the inclusions over different time steps are different.

In some embodiments, the analytical processor may generate a calculated composition based on the change in the composition of the inclusions based on a plurality of time steps output by the composition model. More descriptions of the calculated composition may be found in the relevant descriptions below.

In some embodiments, the analysis processor may generate a trained composition model through training based on a plurality of labeled training samples. For example, the analysis processor may input the plurality of the labeled training samples into an initial composition model, construct a loss function based on labels and an output of the initial composition model, and iteratively update model parameters of the initial composition model based on the loss function. The model training is completed when the loss function of the initial composition model satisfies a preset condition, and the trained composition model is obtained. The preset condition may include the loss function converging, a count of iterations reaching a threshold, or the like.

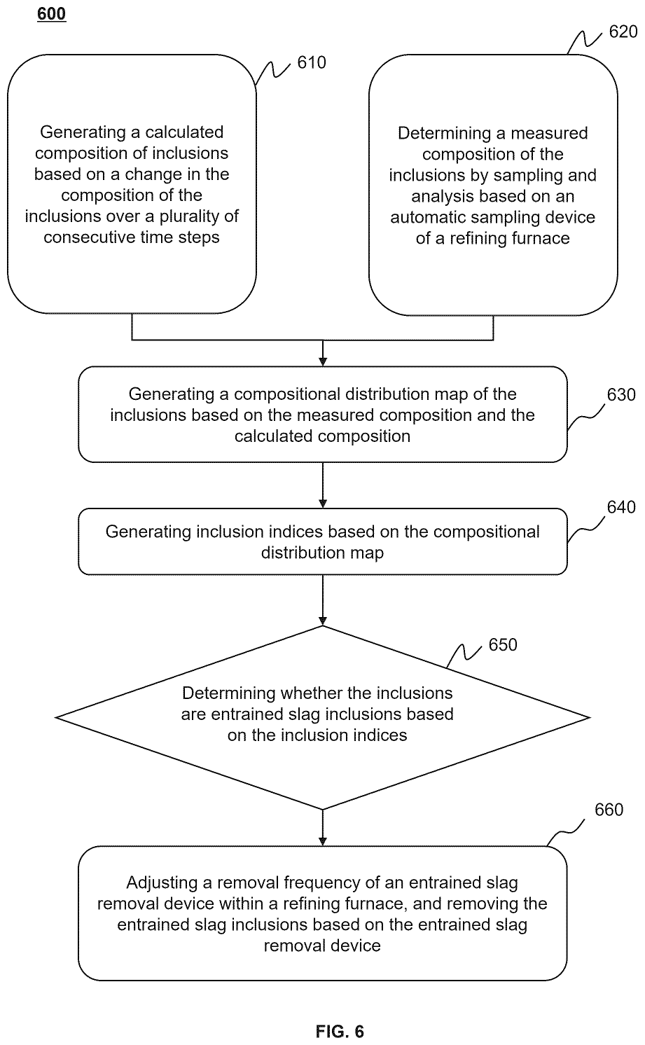

In some embodiments, the training samples may include a sample reaction time, a sample initial composition, a volume of sample inclusions, a mass transfer coefficient of the component M in sample molten steel, and an average size of the sample inclusions at a first historical moment in historical data. The labels corresponding to the training samples may be a change in a measured composition of the inclusions at second historical moments in the historical data. The first historical moment precedes the second historical moments, the first historical moment and the second historical moments are historical moments, and an interval between the first historical moment and each second historical moment is a time step. More descriptions of the measured composition may be found in FIG. 6 and its related descriptions.

In some embodiments, the analysis processor may generate a training sample based on historical sampling analysis data from the first historical moment, and a label corresponding to the training sample based on the measured composition corresponding to the historical sampling analysis data from the second historical moment.

In some embodiments, the analysis processor may divide the training samples into a training set and a validation set, and train and validate the composition model based on the training set and the validation set, respectively. Validation refers to inputting specified training samples (e.g., the training samples in the validation set) into the composition model and verifying that the output of the composition model satisfies a usage requirement.

In some embodiments, the preset condition may further include that the predicted change in the composition of the inclusions output from the composition model is within a validation tolerance range.

The validation tolerance range is a numerical range for determining whether a result output by a composition model is qualified. In some embodiments, the validation tolerance range may be pre-set and determined manually and entered into the analytical terminal.

In some embodiments, the analysis processor may generate the validation tolerance range based on a dynamic sampling cycle of the historical sampling analysis data. The shorter the dynamic sampling cycle is, the smaller the validation tolerance range may be. For example, the analysis processor may determine the validation tolerance range by querying a validation tolerance range table. The validation tolerance range table includes different dynamic sampling cycles and validation tolerance ranges corresponding to the different dynamic sampling cycles.

In some embodiments, the validation tolerance range table may be determined based on sampling analysis data between different dynamic sampling cycles in historical production data. For example, the analysis processor may determine reasonable upper limits and reasonable lower limits of fluctuations in the measured composition based on the fluctuations of a plurality of measurements corresponding to different dynamic sampling cycles and determine, for each dynamic sampling cycle, the reasonable upper limit and lower limit of the fluctuations in the measured composition as the validation tolerance range corresponding to the dynamic sampling cycle, thereby constructing the validation tolerance range table. The shorter the dynamic sampling cycle is, the smaller the range between the reasonable upper limit and lower limit of the fluctuations in the measured composition is, thus the smaller the validation tolerance range is.

More descriptions of the dynamic sampling cycle and the measured composition may be found in FIG. 6 and the related descriptions thereof.

In some embodiments, the analysis processor inputs the validation set into the composition model, and in response to determining that the change in the composition of the inclusions output from the composition model is within the validation tolerance range, the output result is determined to be qualified. For example, if the change in the composition of the inclusions is the rate of change in the composition of the inclusions in the molten steel in one time step, the validation tolerance range may be (0.2-0.5, 0.01-0.05, 0.1-0.4, 0-0.1), indicating the validation tolerance range for the change in the composition of CaO, Al₂O₃, MgO, and La₂O₃ in the inclusions over one time step is 0.2-0.5wt %/s, 0.01-0.05wt %/s, 0.1-0.4wt %/s, and 0-0.1wt %/s, respectively. If the predicted change in the composition of CaO, Al₂O₃, MgO, and La₂O₃ in the inclusions over one time step output by the composition model is 0.4wt %/s, 0.03wt %/s, 0.24wt %/s, 0.06wt %/s, respectively, which are all within the validation tolerance range, it indicates that the model output results are qualified.

In some embodiments, if a ratio of a count of qualified samples to a total count of samples in the validation set is greater than a preset ratio threshold, it may be that the trained composition model satisfies the preset condition. The preset ratio threshold may be determined based on prior experience. For example, the preset ratio threshold may be 0.85, 0.9, 0.95, or the like.

In some embodiments of the present disclosure, the change in the composition of the inclusions over a time step can be efficiently and accurately determined based on the composition model. Furthermore, the prediction result of the composition model may be used to validate the accuracy of the calculation results of the equations, which in turn ensures the reliability of a determination structure of the method.

In 150, determine whether the inclusions are the entrained slag inclusions.

The entrained slag inclusions refer to refined slag inclusions that are mixed into the molten steel. More descriptions of the molten steel and the inclusions may be found in related descriptions of FIG. 1.

The refined slag refers to a solid material formed by mixing a raw material for steel refining in a certain proportion, melting the mixture into a liquid state at a high temperature above the slag's melting point in a specialized device, and then cooling and crushing the molten material. Main components of the refined slag include, but are not limited to, CaO, Al₂O₃, SiO₂, and MgO.

In some embodiments, the analytical processor may determine whether the inclusions in the molten steel are the entrained slag inclusions based on the magnitude of the value of an inclusion index. For example, in response to determining that a CaO index is in a range of 0.52-1, an Al₂O₃ index is in a range of 0.56-2.37, an MgO index is in a range of 0.15-1, and a La₂O₃ index is in a range of 0.29-1, the analytical processor may determine that the inclusions are the entrained slag inclusions.

The inclusion index refers to a ratio of a content of the composition of the inclusions to a content of the initial composition of the inclusions. In some embodiments, the inclusion indices include a CaO index, an Al₂O₃ index, an MgO index, and a La₂O₃ index. More descriptions of the initial composition may be found in related descriptions above.

In some embodiments, as the reaction between the molten steel and the inclusions proceeds, the reaction rate varies with different reaction times, resulting in different changes in the composition of the inclusions, consequently, different inclusion indices for different reaction times. More descriptions of the reaction time may be found in related descriptions below. Understandably, the inclusion index varies from one time step to another due to the different changes in the composition of the inclusions from one time step to another. Thus, in response to a plurality of CaO indices all in the range of 0.52-1, a plurality of Al₂O₃ indices all in the range of 0.56-2.37, a plurality of MgO indices all in the range of 0.15-1, and a plurality of La₂O₃ indices all in the range of 0.29-1, the analytical processor may determine the inclusions to be the entrained slag inclusions.

The content of the composition of the inclusions refers to a compositional content of a substance in the inclusions. For example, the compositional content of the inclusions may be (20 wt %, 15 wt%, 32 wt %, 5 wt%), which may indicate that the compositional content of CaO, Al₂O₃, MgO, and La₂O₃ in the inclusions is 20 wt %, 15 wt%, 32 wt %, and 5 wt %, respectively.

In some embodiments, the compositional content of the inclusions may include a calculated composition and a measured composition. More descriptions of the calculated composition and the measured composition may be found in related descriptions below.

In some embodiments, the analytical processor may determine the compositional content of the inclusions in a variety of manners based on the change in the composition of the inclusions for the plurality of the time steps determined in operation 140. For example, the analytical processor may analyze the change in the composition of the inclusions for the plurality of the time steps by an integral function, a Riemann function, or the like to determine the compositional content of the inclusions. For example, the analytical processor may generate amounts of change in the composition of the inclusions based on the change in the composition of the inclusions over a plurality of time steps multiplied by the time step, and then generate a total amount of change in the composition of the inclusions over the plurality of the time steps by summing the amounts of change in the composition of the inclusions over the plurality of the time steps. Further, the analytical processor may generate the calculated composition of the inclusions over a current time step based on the initial composition and a total amount of change in the composition of the inclusions over the current time step. More descriptions of the composition of the inclusions may be found in the description of the initial composition above.

In some embodiments, the analytical processor may determine the compositional content of the inclusions by sampling and analysis. More descriptions of the sampling and analysis may be found in related descriptions below.

In some embodiments, the inclusion indices may include a CaO index, an Al₂O₃ index, an MgO index, and a La₂O₃ index.

In some embodiments, a value range of the CaO index is 0.52 to 1, a value range of the Al₂O₃ index is 0.56 to 2.37, a value range of the MgO index is 0.15 to 1, and a value range of the La₂O₃ index is 0.29 to 1.

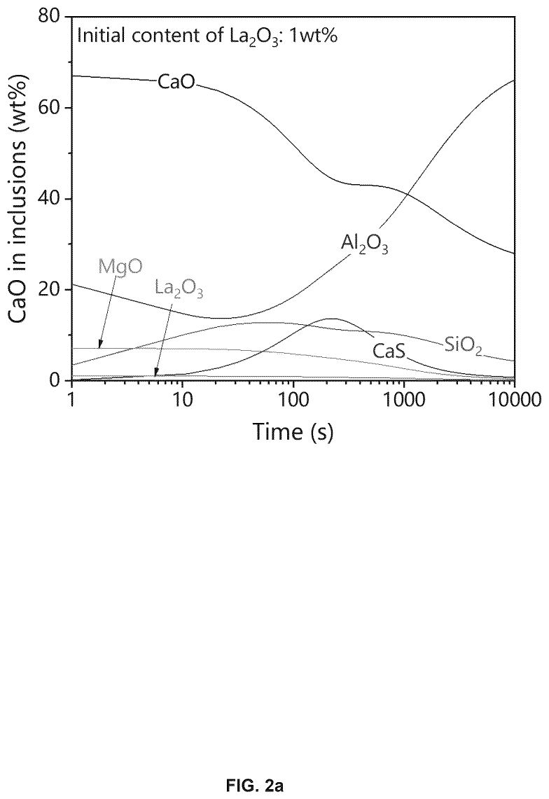

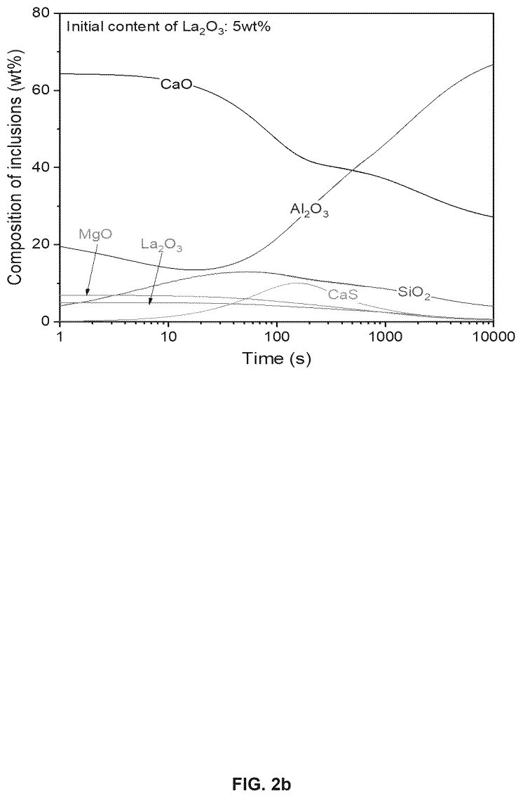

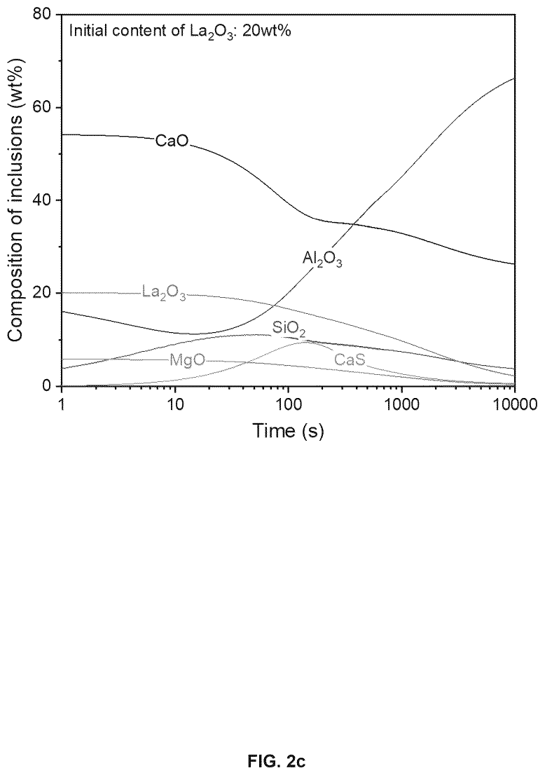

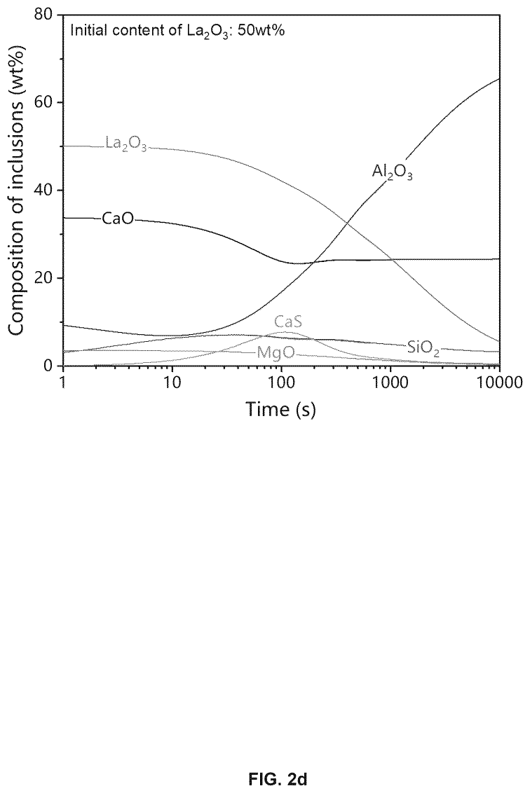

FIGS. 2a to 2d show evolution of the composition of entrained slag inclusions with an initial La₂O₃ content of 1%, 5%, 20%, and 50%, respectively, after different reaction times with the molten steel.

In some embodiments, as the entrained slag inclusions continue to react with the molten steel, the content of CaO, MgO, and La₂O₃ in the entrained slag inclusions gradually decreases. The content of Al₂O₃ decreases and then increases, while the content of SiO₂ and CaS increases and then decreases. The reaction between the molten steel and the entrained slag inclusions also varies with the reaction time. During the first few tens of seconds after the entrained slag inclusions enter the molten steel, Al₂O₃ in the entrained slag inclusions is reduced by dissolved silicon ([Si]) in the molten steel. When SiO₂ in the entrained slag inclusions reaches a peak, dissolved aluminum ([Al]) and oxygen ([O]) in the molten steel react to produce Al₂O₃, while CaO in the inclusions is reduced by [Al]. As a result, the Al₂O₃ content of the entrained slag inclusions continues to increase, while the CaO content steadily decreases. During the first 150 seconds, due to a high sulfur content in the molten steel, CaO in the inclusions reacts with [S] in the molten steel to form CaS, resulting in a gradual increase in the CaS content in the entrained slag inclusions. After reaching a peak at around 150 seconds, the CaS content gradually decreases.

In some embodiments, when the initial La₂O₃ content in the refined slag is 1%, the composition of the entrained slag inclusions is similar to that without a tracer test. Initially, the CaO content of the entrained slag inclusions decreases rapidly from an initial content of 67% to 42%. Over time, the rate of decrease in CaO content gradually slows down. After 10,000 seconds of reaction, the CaO content is about 28%.

In some embodiments, due to the reduction reaction of dissolved sulfur [S] in the molten steel with CaO in the inclusions, the CaS content in the entrained slag inclusions gradually rises, and then declines after reaching a peak of 13.6%. This decline is due to the continuous increase in the Al₂O₃ content in the inclusions, which leads to a decrease in the relative content of CaO and CaS, ultimately reducing the CaS content in the inclusions to less than 1%. The SiO₂ content initially increases rapidly from 0.3% to about 13% and then gradually decreases to about 5%. The decrease in the SiO₂ content is slower than the decrease in the CaO content and CaS content because the amount of SiO₂ generated is less than that of Al₂O₃, although some SiO₂ still forms.

The MgO content and the La₂O₃ content decrease throughout the reaction. When the initial La₂O₃ content is increased to 5% and 50%, the reaction between the molten steel and the entrained slag inclusions in a reaction layer remains the same, but the final composition of the reaction layer is slightly different. After 10,000 seconds of reaction, the Al₂O₃ content in the entrained slag inclusions is is 66.13%, 66.73%, 66.79%, and 65.46% for initial La₂O₃ contents of 1%, 5%, 20%, and 50%, respectively. The CaO content is 27.92%, 27.1%, 25.87%, and 24.38%, respectively, with a difference within 10%.

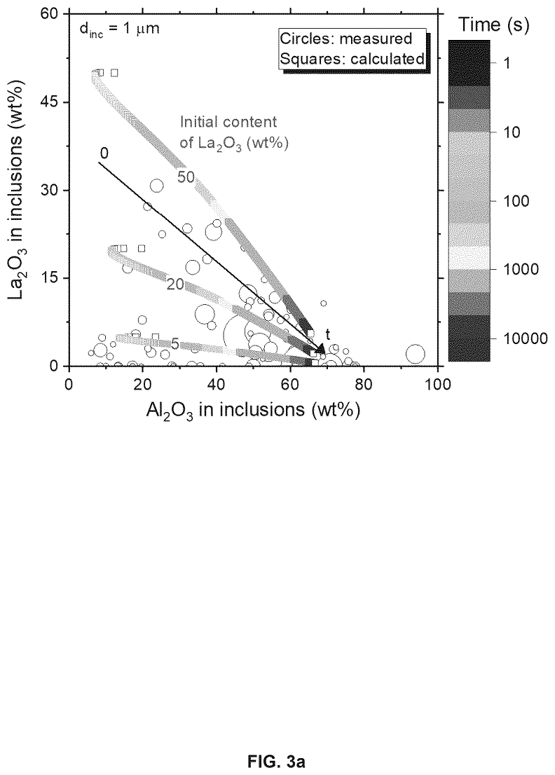

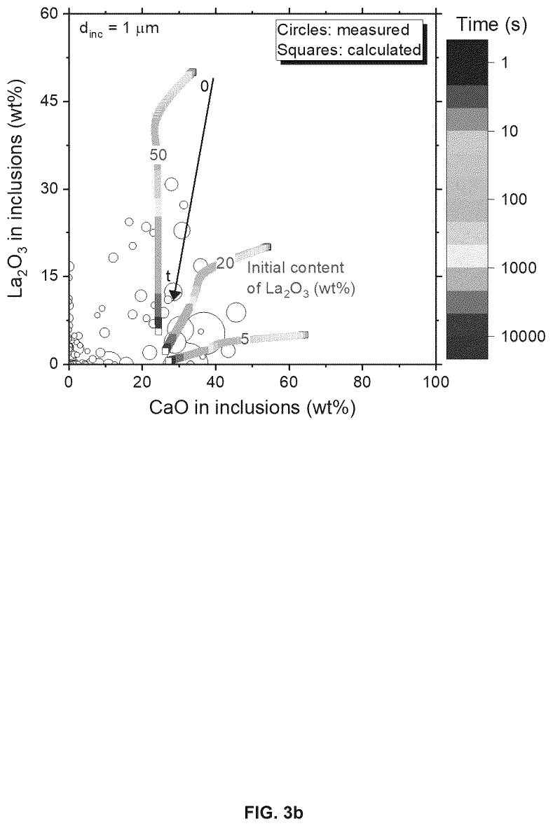

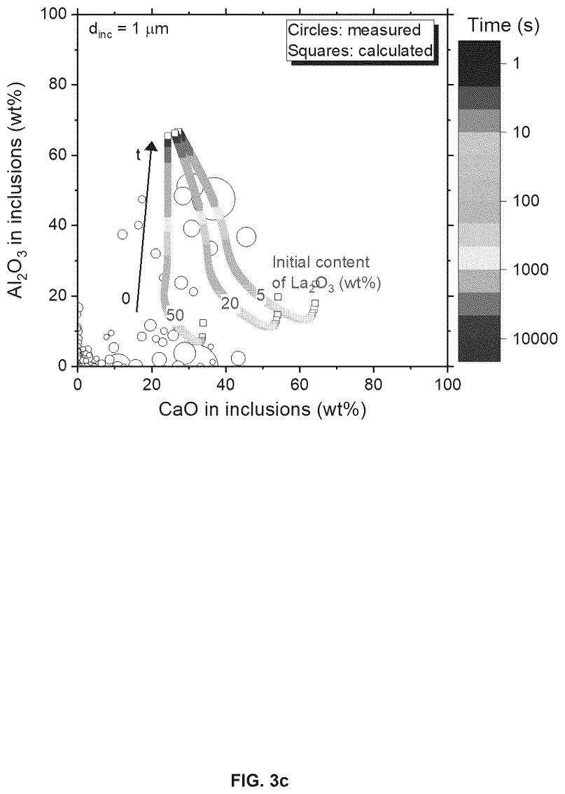

In order to further investigate a compositional distribution of the entrained slag inclusions after reaction with the molten steel, the evolution of the composition of the entrained slag inclusions is generated based on the analytical processor for different initial La₂O₃ contents (e.g., 5%, 20%, and 50%). The resulting compositions were projected onto binary compositional distribution diagrams of La₂O₃—Al₂O₃, La₂O₃—CaO, and CaO—Al₂O₃, as shown in FIGS. 3a to 3c.

In the drawings, the squares represent calculated compositions, while the circles represent measured compositions. The initial La₂O₃ content in the refined slag has a small effect on the final composition of the entrained slag inclusions, but a large effect on the conversion process. The drawings show the compositional distribution of entrained slag inclusions with a diameter of 1 μm after 10,000 seconds of reaction with molten steel. Considering the typical size of the inclusions and their residence time during an actual molten steel refining process, it is likely that the inclusions are located within their respective compositional points at each time point. However, even under an extreme condition of an initial La₂O₃ content of 50%, the Al₂O₃ content after reaction does not exceed 70% and the CaO content is below 20%. The final La₂O₃ content varies greatly and is closely related to the initial La₂O₃ content. Generally, after 3,000 seconds of reaction, the La₂O₃ content is less than 15%.

Comparing the calculated compositions and the measured compositions, it is found that not all inclusions containing La₂O₃ are entrained slag inclusions. In order to determine whether the inclusions are entrained slag inclusions or not, it is necessary to take into account the combined content of CaO, Al₂O₃, and La₂O₃. In some embodiments, almost all tested inclusions satisfy a La₂O₃—Al₂O₃ binary composition requirement for entrained slag inclusions. However, a significant portion of the inclusions fall outside of an expected compositional range of the entrained slag inclusions when considering the CaO content in the inclusions, as shown in FIG. 3b and FIG. 3c.

The obtained kinetic results show that the composition of the entrained slag changes due to reactions with the molten steel. However, due to certain unpredictable factors of the inclusions, such as entrainment duration and residence time in the molten steel, it is almost impossible to determine the reaction time between the coil slag and the molten steel. Therefore, accurately distinguishing between the entrained slag inclusions and inclusions produced by deoxidation reaction is a major challenge.

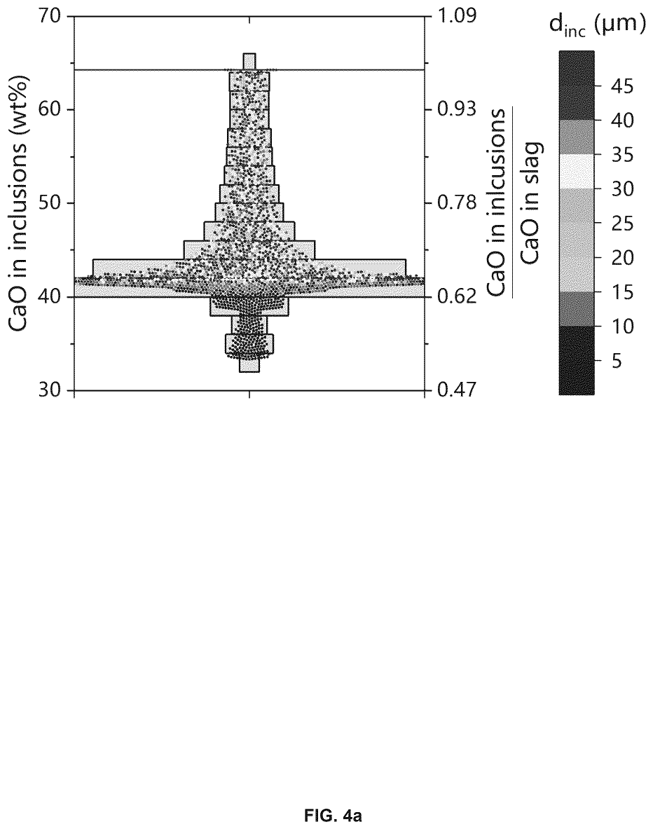

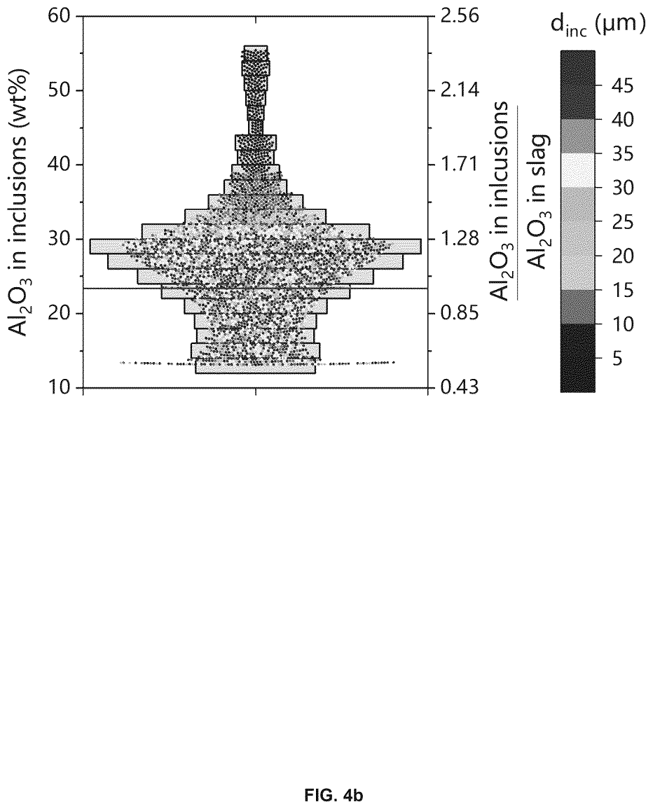

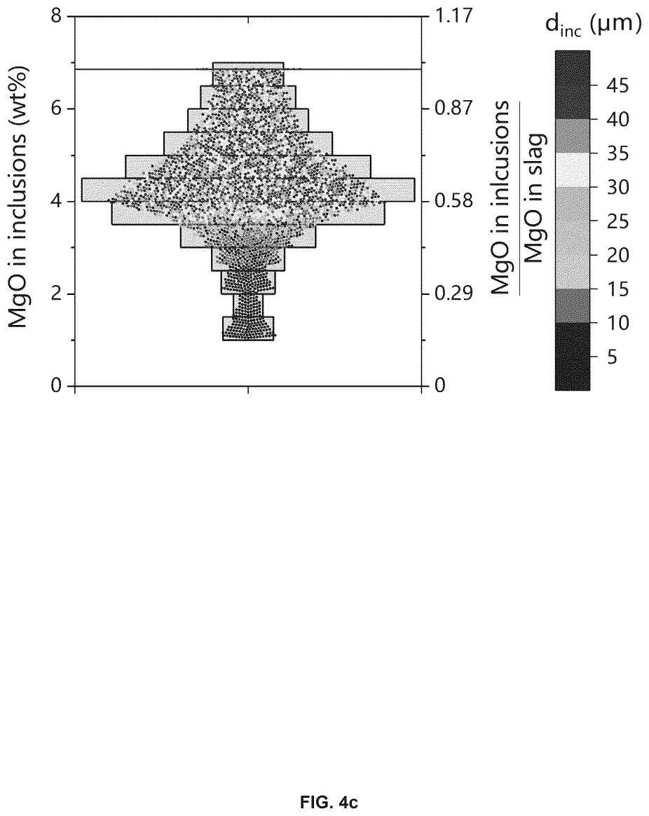

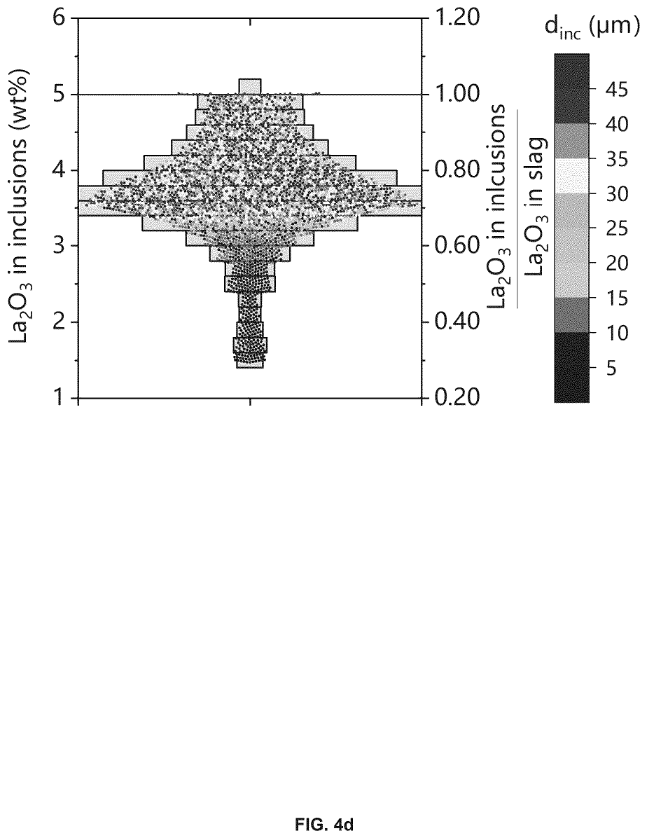

In order to clarify the compositional distribution of the entrained slag, the present embodiment determine compositional transformation using the current model. The initial La₂O₃ content is set to 5%, and the inclusions range in diameter from 1 μm to 50 μm. The composition of the entrained slag inclusions is recorded throughout the determination process to indicate the different residence times of the entrained slag inclusions in the molten steel. All calculations results are then plotted to analyze a probability distribution of the composition of the entrained slag, as shown in FIG. 4a to FIG. 4d. Each point in the drawings corresponds to an inclusion, and the color represents the diameter of the inclusion. The left Y-axis represents the composition of the inclusions, and the right Y-axis represents the ratio of the composition of the entrained slag inclusions to the initial composition of the steel slag, which is referred to as the inclusion index. After reacting with the molten steel, the calculated CaO content in the entrained slag inclusions ranges from 33% to 65%, with the highest content of CaO in the entrained slag inclusions being about 42%. The Al₂O₃ content of the entrained slag inclusions ranges from 13% to 55%, with a maximum distribution probability of about 30%. The MgO content and the La₂O₃ content in the entrained slag inclusions have a similar distribution pattern, and the reaction between the molten steel and the entrained slag inclusions leads to a decrease in the MgO content and the La₂O₃ content. As a result, the MgO content and the La₂O₃ content in the entrained slag inclusions are lower than those in the steel slag.

There is a clear correlation between the composition of the entrained slag inclusions and the diameter of the inclusions, and it is observed that the inclusions with smaller sizes react faster, resulting in a larger difference between a real-time composition of the entrained slag inclusions and the original composition. After reacting with the molten steel, it is found that most of the entrained slag inclusions with a diameter of less than 15 μm has a CaO content of less than 41%, an Al₂O₃ content of more than 32%, an MgO content of less than 3.5%, and a La₂O₃ content of less than 3.3%.

Due to the differences between the entrained slag inclusions and the refined slag, some embodiments of the present disclosure introduce entrained slag inclusion indices to determine whether the inclusions are the entrained slag inclusions. The inclusion index is defined as the ratio of the composition of the inclusions to the initial composition of the inclusions. According to the calculation results of the analytical processor, the criteria for identifying the entrained slag inclusions are as follows: the CaO index in the range of 0.52-1, the Al₂O₃ index in the range of 0.56-2.37, the MgO index in the range of 0.15-1, the La₂O₃ index in the range of 0.29-1. If the composition an inclusion in the molten steel satisfy the above range of the four indices, the inclusion may be identified as an entrained slag inclusion.

For example, the analytical processor may generate the calculated composition of the inclusions based on the change in the composition of the inclusions over a plurality of the time steps, determine the measured composition of the inclusions through sampling and analysis, generate a compositional distribution diagram of the inclusions based on the measured composition and the calculated composition, generate inclusion indices based on the compositional distribution diagram, and determine whether the inclusions are entrained slag inclusions based on the inclusion indices. More descriptions of determining whether the inclusions are entrained slag inclusions may be found in FIG. 6 and its related descriptions.

Some embodiments of the present disclosure, by proposing reasonable criteria for identifying entrained slag, it can be clearly determined whether inclusions in the molten steel originate from refined slag, which helps to trace the source of the inclusions, thereby providing a clear direction for regulating the inclusion content in molten steel during metallurgical production. Consequently, appropriate industrial measures can be taken to adjust the steelmaking process, control the occurrence of entrained slag inclusions, reduce the count of entrained slag inclusions in molten steel, improve process levels, and enhance the quality of steel products.

It should be noted that the above descriptions of the process 100 are intended to be exemplary and illustrative only and do not limit the scope of the present disclosure. For a person of ordinary skill in the art, various modifications and variations may be made to the process 100 under the guidance of the present disclosure. However, these modifications and variations remain within the scope of the present disclosure.

FIG. 6 is a flowchart illustrating an exemplary process of a method for processing entrained slag inclusions in steel with deoxidized calcium with fixed aluminum according to some embodiments of the present disclosure. As shown in FIG. 6, process 600 includes the following operations. In some embodiments, process 600 may be executed by an analytical terminal.

In 610, generating a calculated composition of inclusions based on a change in the composition of the inclusions over a plurality of consecutive time steps.

The calculated composition refers to compositional contents of different components in the inclusions, determined by Equations (1) to (14). In some embodiments, the calculated composition may include the compositional content of CaO, Al₂O₃, SiO₂, and MgO in the inclusions.

In some embodiments, the analytical processor may generate the calculated composition of the inclusions based on the change in the composition of the inclusions over a plurality of consecutive time steps. The process of generating the calculated composition is similar to the process of generating the compositional content of the inclusions, and is not described herein.

In some embodiments, the analytical processor may store the calculated composition into an analytical data storage of an analytical terminal.

In 620, determining a measured composition of the inclusions by sampling and analysis based on an automatic sampling device of a refining furnace.

The measured composition refers to measured compositional contents of the different components in the inclusions. The data format for the measured composition is similar to the data format of the calculated composition, which is not repeated here.

In some embodiments, the analytical processor may determine, based on the automatic sampling device of the refining furnace, the measured composition of the inclusions through sampling and analysis based on the automatic sampling device of the refining furnace, and store the measured component in the analysis data storage of the analysis terminal.

The sampling and analysis refer to a process of extracting a portion of samples from the molten steel for analysis. The sampling and analysis may include periodic sampling and dynamic sampling. The periodic sampling refers to a sampling process in which sampling is performed at regular time intervals, while the dynamic sampling refers to a sampling process in which time intervals between each sampling are not fixed.

In some embodiments, the analytical processor may generate a dynamic sampling cycle and issue a sampling instruction to the refining furnace to control the automatic sampling device of the refining furnace to collect an inclusions sample in accordance with the sampling instruction. The analytical processor may analyze an inclusion sample, determine the composition of the inclusions, and store the composition in the analytical data storage.

More descriptions of the analytic processor and the analytical data storage may be found in FIG. 1 and its related descriptions.

The refining furnace is a vessel that holds the molten steel, the refined slag, and the inclusions. The refining furnace may be a ladle furnace, an electric furnace, or the like.

The automatic sampling device is a device for automatic sampling. The automatic sampling device may be mechanically coupled to the refining furnace for extracting a sample of the molten steel and the inclusions from the refining furnace. In some embodiments, the automatic sampling device may include a robotic arm.

The analysis refers to analyzing the compositional content of the inclusion sample. For example, the analytic processor may analyze the inclusion sample using an analytical instrument to generate the compositional content of the inclusions using techniques such as atomic absorption, X-ray diffraction, or the like. More descriptions of the compositional content of the inclusions may be found in the related descriptions above

The inclusion sample is a material that contains the molten steel and inclusions. The inclusion sample may be obtained from the refining furnace by the automatic sampling device. For example, the analytic processor may control the robotic arm of the automatic sampling device to take a portion of a mixture of partial molten steel and inclusions from the refining furnace, which is then cooled to obtain inclusion sample.

The dynamic sampling cycle refers to sequence data consisting of time intervals between different batches of samples in dynamic sampling. For example, (20, 50, 180, 500) may indicate that the time intervals between different sequences of sampling in 5 dynamic samplings are 20s, 50s, 180s, and 500s, respectively. Wherein, 20s is the time interval between a first sampling and a second sampling, 50s is the time interval between the second sampling and a third sampling, 180s is the time interval between the third sampling and a fourth sampling, and 500s is the time interval between the fourth sampling and a fifth sampling.

In some embodiments, the analytic processor may select a plurality of sampling time points within a total sampling period. The analytic processor may determine the dynamic sampling cycle based on the plurality of sampling time points and store the dynamic sampling cycle in the analytical data storage. The sampling time points may characterize representative time points at which reactions occur between the molten steel and inclusions inside the refining furnace.

In some embodiments, the analytic processor may divide the total sampling period into a plurality of consecutive time steps of equal size, thereby determining the dynamic sampling cycle. In some embodiments, the faster the reaction rate, the fewer a count of the time steps, and consequently, the shorter the sampling cycle. The reaction rate may be determined by the analytic processor based on historical production records.

In some embodiments, the analytic processor may determine the count of the time steps between sampling points by querying a sampling table. The sampling table includes reaction times and the counts of time steps corresponding to reaction times.

In some embodiments, the analytic processor may determine the sampling table based on historical production data. For example, the analytic processor may determine reaction rates corresponding to different reaction times based on a plurality of historical production records, and designate a smallest time interval in which a change in the reaction rate reaches a preset change threshold as the sampling cycle, thereby determining the count of the time steps. The preset change threshold is a manually pre-determined threshold for the magnitude of change in the reaction rate. Setting the minimum time interval for the change in the reaction rate to reach the preset change threshold may avoid performing further sampling when the change in the reaction rate is too small, thereby avoiding the waste of manpower and resources. The faster the reaction rate is, the greater the amount of change in the reaction rate within a time step, and the greater the value of the change in the composition of different components in the inclusions.

The total sampling period is the reaction time of the molten steel and the inclusions, i.e., the time period during which sampling may be performed. For example, the total sampling period may range from a reaction time of 0 seconds to 10000 seconds. In some embodiments, the total sampling period may be determined based on historical production data. For example, the analytic processor may designate a duration from a start time to an end time of a production cycle in the historical production data as the total sampling period.

The sampling time points refer to predetermined points in time at which dynamic sampling is planned to be performed. The sampling time points are within the total sampling period.

In some embodiments, the analytic processor may determine compositional change mutation points based on the change in the composition of the inclusions predicted by a composition model at different time steps, and designate the compositional change mutation points as the sampling time points. For example, the analytic processor may predict changes (e.g., 13.1, 13.3, 13.7, 18.5, 18.8, 19.2, 28.3, and 28.8) in the composition of CaO at different time steps based on the composition model and determine 18.5 and 28.3 as the compositional change mutation points, thereby designating the time points corresponding to 18.5 and 28.3 as the sampling time points.

More descriptions of the composition model may be found in FIG. 1 and its related notes above.

The compositional change mutation points refer to time points at which the compositional change of different components of the inclusions undergo sudden changes.

For example, the analytic processor may determine the compositional change mutation points in a variety of ways. For example, the analytic processor may set a preset change threshold and identify time points when the compositional change is greater than or equal to the preset change threshold as the compositional change mutation points. More descriptions of the preset change threshold may be found in the related descriptions above.

In some embodiments of the present disclosure, by determining the plurality of sampling time points, a more reasonable dynamic sampling cycle can be generated for guiding the sampling and analysis. By determining the sampling time points based on the compositional change mutation points, the workload of manually calculating the sampling cycle can be reduced, thereby achieving automated sampling and analysis and minimizing human error.

In 630, generating a compositional distribution diagram of the inclusions based on the measured composition and the calculated composition.

A compositional distribution diagram of inclusions is a graph of data reflecting the compositional content of different components of the inclusions at different reaction times. Compositional distribution diagrams of the inclusions are shown in FIG. 3a˜FIG. 3c. In some embodiments, the analytic processor may generate the compositional distribution diagram of the inclusions based on data of the calculated composition and the measured composition at different times.

In 640, generating inclusion indices based on the compositional distribution diagram.

In some embodiments, the analysis processor may determine the distribution of components of the slag inclusions of different sizes in the molten steel based on the component distribution diagram, as shown in FIGS. 4a to 4d. Subsequently, based on the component distribution, the analysis processor may determine value ranges of the inclusion indices indicating slag inclusions in the molten steel. For example, for each of the inclusion indices, the analysis processor may determine a minimum value and a maximum value of the inclusion index when the inclusions are determined to be the entrained slag inclusions in a plurality of sampling analyses, thereby determining the value range of the inclusion index.

The inclusion indices may include a CaO index, an Al₂O₃ index, an MgO index, and a La₂O₃ index. More descriptions of the inclusion indices may be found in FIG. 1 and the related descriptions above.

In some embodiments, the value ranges of the CaO index, the Al₂O₃ index, the MgO index, and the La₂O₃ index correlate to a difference between the calculated composition and the measured composition. The analytic processor may generate an adjustment instruction based on the difference between the calculated composition and the measured composition, adjust the value ranges of the CaO index, the Al₂O₃ index, the MgO index, the La₂O₃ index based on the adjustment instruction, and update stored data in the analytical data storage. The adjustment instruction may reflect different adjustment ranges.

The difference between the measured composition and the measured composition refers to an absolute value of a difference between the measured composition and the calculated composition corresponding to the molten steel and the entrained slag inclusions at a same sampling time point. For example, if the measured composition determined by sampling and analysis at a sampling time point A is m, and the calculated composition determined by calculation is n, the analytic processor may generate the difference between the measured composition and the measured composition by determining the absolute value of the difference between m and n, i.e., |m-n|.

The adjustment instruction is an instruction issued by the analytic processor to adjust the value range of the inclusion indices. For example, the adjustment instruction may include adjustment ranges for the inclusion indices.

An adjustment range refers to a magnitude of the adjustment to the value range of an inclusion index. For example, an adjustment range of CaO-(−20%, +20%) indicates lowering the lower limit of the value range of CaO index by 20%, and increasing the upper limit of the value range of CaO index by 20%.

In some embodiments, the adjustment instruction includes an adjustment instruction for the CaO index, an adjustment instruction for the Al₂O₃ index, an adjustment instruction for the MgO index, and an adjustment instruction for the La₂O₃ index. The adjustment instruction for the CaO index is positively correlated to a difference between a calculated composition and a measured composition of CaO; the adjustment instruction for the Al₂O₃ index is positively correlated to a difference between a calculated composition and a measured composition of the Al₂O₃; the adjustment instruction for the MgO index is positively correlated to a difference between the calculated compositions and the measured compositions of the MgO; and the adjustment instruction for the La₂O₃ index is positively correlated to a difference between the calculated compositions and the measured compositions of the La₂O₃.

In some embodiments, the analytic processor may generate the adjustment instruction based on a weighted average of the difference between the calculated composition of CaO and the measured composition of CaO, a weighted average of the difference between the calculated composition of the Al₂O₃ and the measured composition of the Al₂O₃, a weighted average of the difference between the calculated composition of the MgO and the measured composition of the MgO, and a weighted average of the difference between the calculated composition of the La₂O₃ and the measured composition of the La₂O₃, wherein weighting coefficients are related to the dynamic sampling cycle.

In some embodiments, for a component, the analytical processor may set the weight coefficients for the differences between the measured composition and the calculated composition at different sampling time points, and determine the weighted average of the differences through weighting. More descriptions of the dynamic sampling cycle may be found in the related descriptions above.

For example, for a component, the weighted average may be generated based on Equation (15):

$$Q = {A}_{1} \times {q}_{1} + {A}_{2} \times {q}_{2} + … + {A}_{n} \times {q}_{n} \qquad ( 15 )$$

wherein Q denotes the weighted average, A₁, A₂, . . . , and Aₙ denotes the difference between the measured composition and the calculated composition at sampling time point 1, sampling point 2, . . . , and sampling time point n, respectively, q₁, q₂, . . . , and qₙ are the weighting coefficients.

Understandably, the weighting coefficients corresponding to different sampling time points may be the same or different. When sampling time intervals between different sampling time points are the same, the weighting coefficients corresponding to the different sampling time points may be the same; when the sampling time intervals between different sampling time points are different, the weighting coefficients corresponding to the different sampling time points may be different.

In some embodiments, for a sampling time point, the corresponding weighting coefficient may be determined based on the sampling time interval between that sampling time point and a previous sampling time point.

In some embodiments, because the shorter the dynamic sampling cycle is, the smaller a validation tolerance range for judge whether an output result of the compositional model is qualified, and thus the higher the accuracy of the output result of the trained compositional model is. When the dynamic sampling cycle is shorter, the difference between the measured composition and the calculated composition at the corresponding sampling time point is more accurate. Therefore, the shorter the sampling interval is, the higher the weighting coefficient is. For example, the weighting coefficient=(1-sampling interval/total sampling period)/(total count of sampling time points n-1). More descriptions of the composition model, the validation tolerance range, and the total sampling period may be found in the related descriptions above. By applying the above embodiments, the analytical terminal can ensure higher accuracy of the weighted average, which is more consistent with reality.

In some embodiments, the analytical processor may query an adjustment table to determine the adjustment range for the CaO index based on the weighted average of the difference between the calculated composition and the measured composition of CaO; determine the adjustment range for the Al₂O₃ index based on the weighted average of the difference between the calculated composition and the measured composition of Al₂O₃; determine the adjustment range for the MgO index based on the weighted average of the difference between the calculated composition and the measured composition of MgO; and determine the adjustment range for the La₂O₃ index based on the weighted average of the difference between the calculated composition and the measured composition of La₂O₃.

In some embodiments, the adjustment table may include weighted averages of the different components and adjustment ranges for the indices corresponding to the components. In some embodiments, the analysis processor may determine the adjustment range corresponding to each component based on an average value of a compositional range of the slag inclusions determined by further sampling and analysis based on historical production data of the refining furnace, and then generate the adjustment table.

For example, if the value range of the CaO index is initially 0.52-1, and it is determined that the compositional range of the entrained slag inclusions is 0.40-1.2 by further manual sampling and analysis, then the adjustment range for the CaO index may be determined based on calculation. For example, the analysis processor may determine the weighted average of the differences as follows: [|(0.39−0.52)/0.52|+(1.2−1)/1]/2=0.225. Therefore, the adjustment range for the CaO index determined by the analysis processor may be (−0.225, 0.225), and an actual value range of the CaO index may be determined by calculating 0.52×(1−0.225) and 1×(1+0.225), resulting in the actual value range of 0.403-1.225 for the CaO index. The analysis processor can generate the adjustment table based on a plurality of weighted averages of differences and the value ranges for the corresponding inclusion indices.

In some embodiments of the present disclosure, by considering the magnitude of the difference between the calculated composition and the measured composition, the analysis processor can adjust the value ranges of different inclusion indices, ensuring that the determination of the entrained slag inclusions is more accurate and realistic, thereby reducing an interference caused by occasional fluctuations in production parameters of the refining furnace on the determination of the inclusions.

In 650, determining whether the inclusions are the entrained slag inclusions based on the inclusion indices.

In some embodiments, if the inclusion indices of inclusions in the molten steel falls within the corresponding value range of the inclusion indices, the inclusions in the molten steel are determined to be entrained slag inclusions.

More descriptions of the inclusion indices and the value range of the inclusion indices may be found in FIG. 1 and the related descriptions thereof.

In some embodiments, responsive to determining that the inclusions are the entrained slag inclusions, the analytical processor may perform operation 660.

In 660, adjusting a removal frequency of an entrained slag removal device within the refining furnace and removing the entrained slag inclusions based on the entrained slag removal device.

The entrained slag removal device refers to a device for removing the entrained slag from the molten steel. In some embodiments, the entrained slag removal device may include a gas purge device, an electromagnetic filter device, or the like.

In some embodiments, the analytical processor may determine the removal frequency of the entrained slag removal device by querying an entrained slag removal table. The entrained slag removal table includes different inclusion indices, reaction times, and removal frequencies of the entrained slag removal device corresponding to the different inclusion indices and reaction times.

The analytical processor may determine the entrained slag removal table based on historical production records. For example, the analytical processor may, based on different inclusion indices and different reaction times from historical production records, determine minimum removal frequencies corresponding to subsequent steel without inclusion quality issues as the removal frequencies in the entrained slag removal table.

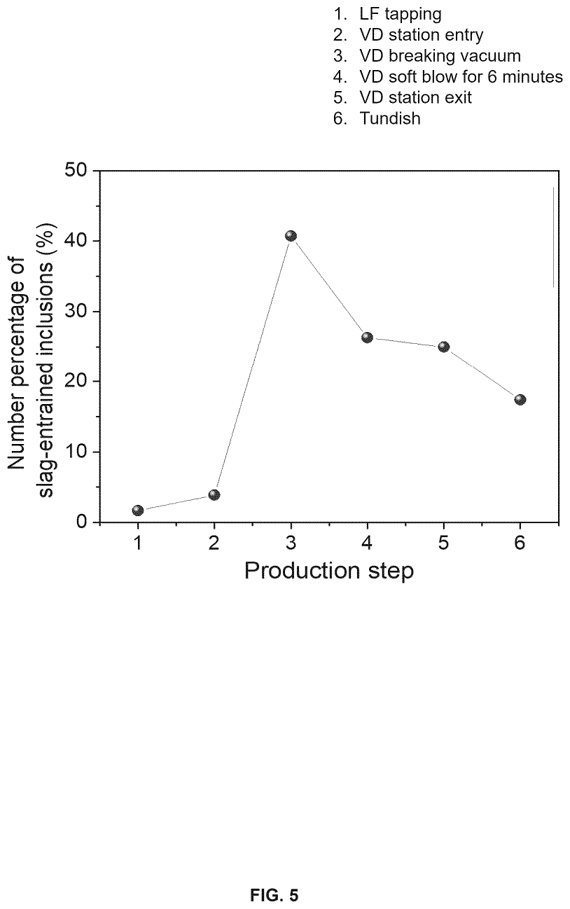

In some embodiments, the analytical processor may determine the range values of the inclusion indices for the entrained slag inclusions in the molten steel processed with aluminum deoxidation calcium by determining the CaO index, the Al₂O₃ index, the MgO index, and the La₂O₃ index of the entrained slag inclusions. Further, the analytical processor may statistically determine a proportion of the entrained slag inclusions in the molten steel by comparing and analyzing the composition of the inclusions in the molten steel obtained by dynamic sampling analysis from different stages of aluminum deoxidation calcium-treated steelmaking, as shown in FIG. 5.

In some embodiments, the proportion of large-size entrained slag inclusions in a molten steel refining process increases rapidly to about 40% due to an intense steel slag reaction. During a steelmaking process, as the large-size entrained slag inclusions continuously float up and the removal frequency of the entrained slag removal device is dynamically adjusted to remove the entrained slag inclusions, the proportion the entrained slag inclusions decreases to about 20%.

In some embodiments of the present disclosure, by determining the calculated composition and the measured composition of inclusions corresponding to a same reaction time point, and then determining whether the inclusions are entrained slag inclusions based on the calculated composition and the measured composition, reasonable adjustments may be made to the removal frequency of the entrained slag removal device to meet quality requirements of steel production, avoid different degrees of inclusion distribution in produced steel, and ensure the stability of steel quality.

Having thus described the basic concepts, it may be rather apparent to those skilled in the art after reading this detailed disclosure that the foregoing detailed disclosure is intended to be presented as illustrative example and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

As another example, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This way of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameter set forth in the written description and attached claims are approximations that may vary depending upon the desired properties sought to be obtained by a particular embodiment. In some embodiments, the numerical parameter should be construed in light of the number of reported significant digits and by applying ordinary rounding techniques. Notwithstanding that the numerical ranges and parameter setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrating of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A method for processing entrained slag inclusions in steel with deoxidized calcium with fixed aluminum, wherein:
an initial composition of inclusions is the same as a composition of a refined slag, including CaO, Al₂O₃, SiO₂, MnO, MgO, CaS, and an added tracer La₂O₃, and a reaction Equation (1) is shown as below;
$$x [ M ] + y [ O ] = ( {M}_{x} {O}_{y} ) \qquad ( 1 )$$
wherein M denotes any one of elements of calcium (Ca), aluminum (Al), silicon (Si), manganese (Mn), magnesium (Mg), or lanthanum (La), O denotes an element of oxygen (O) or an element of sulfur (S), and x and y are positive integers;
the inclusions are in a liquid state and exist in molten steel in a spherical shape, a size of the inclusions remains constant during a reaction process, and for a chemical reaction shown in the Equation (1), a change in Gibbs free energy is generated according to an Equation (2):
$$Δ G = Δ {G}^{⊖} + RTln \frac{{a}_{{M}_{x}^{*} {O}_{y}}}{{a}_{M}^{* x} \cdot {a}_{O}^{* y}} \qquad ( 2 )$$
wherein ΔG denotes the change in Gibbs free energy of the chemical reaction, unit in J/mol, ΔG^{Θ} denotes a standard change in Gibbs free energy, unit in J/mol, R denotes a gas constant, unit in J/(mol-K), T denotes a temperature of the chemical reaction, unit in K, a denotes an activity, and * represents an interface between the inclusions and the molten steel;
an activity of the component M is generated through Equations (3) and (4):
$${a}_{M} = {f}_{M} \cdot [ M ] \qquad ( 3 )$$
$${lgf}_{M} {\sum }_{j} {e}_{M}^{j} [ j ] + {\sum }_{j} {\sum }_{k} {r}_{M}^{j , k} [ j ] [ k ] \qquad ( 4 )$$
wherein a_{M} denotes the activity of the component M, ƒ_{M} denotes an activity coefficient of the component M, [M] denotes a mass fraction of the component M in the molten steel, unit in %, e denotes a first-order activity interaction coefficient, r denotes a second-order activity interaction coefficient, and components of the molten steel include Fe—C—Si—Mn—P—S—Ca—Al—Mg—O, and [j] and [k] denote mass fractions of a j-th component and a k-th component of the molten steel respectively, unit in %, j and k are positive integers;
an activity of a molecular component MₓO_{y} is generated based on an ion and molecule coexistence theory (IMCT), through Equations (5) and (7), and an activity of an ionic component MₓO_{y} is generated through Equations (6) and (7):
$${a}_{{M}_{x} {O}_{y}} = \frac{{N}_{{M}_{x} {O}_{y}}}{{N}_{t}} \qquad ( 5 )$$
$${a}_{{M}_{x} {O}_{y}} = \frac{( x + y ) {N}_{{M}_{x} {O}_{y}}^{′}}{{N}_{t}} \qquad ( 6 )$$
$${N}_{t} = \sum {N}_{{M}_{x} {O}_{y}} + \sum ( x + y ) {N}_{{M}_{x} {O}_{y}}^{′} \qquad ( 7 )$$
wherein NMₓO_{y} denotes a molar amount of the molecular component MₓO_{y}, unit in mol, N_{M}_{ₓ}_{O}_{_{y}}′ denotes a molar amount of the ionic component MₓO_{y}, unit in mol, and Nₜ denotes a total molar amount of all components of the molten steel, including simple compounds and complex compounds generated by the simple compounds, unit in mol;
mass transfer flux equations for the component M in the molten steel and the component MₓO_{y} in the inclusions within the interfacial layer are:
$${J}_{M} = - \frac{{ρ}_{st} {k}_{M}}{100 {M}_{M}} ( {[ M ]}^{b} - [ M ] \text{*)} \qquad ( 8 )$$
$${J}_{( {M}_{x} {O}_{y} )} = - \frac{{ρ}_{inc} {k}_{{M}_{x} {O}_{y}}}{100 {M}_{{M}_{x} {O}_{y}}} ( {[ {M}_{x} {O}_{y} ]}^{b} - [ {M}_{x} {O}_{y} ] \text{*)} \qquad ( 9 )$$
wherein J_{M} denotes a mass transfer flux of the component M in the molten steel, unit in mol/(m2·s), J_{(M}_{ₓ}_{O}_{_{y}}₎ denotes a mass transfer flux of the component MₓO_{y} in the inclusions, unit in mol/(m2·s), ρₛₜ denotes a density of the molten steel, unit in kg/m3, ρ_{inc} denotes a density of the inclusions, unit in kg/m3, k_{M} denotes a mass transfer coefficient of the component M in the molten steel, unit in m/s, k_{M}_{ₓ}_{O}_{_{y}} denotes a mass transfer coefficient of the component MₓO_{y} in the inclusions, unit in m/s, M_{M} denotes a molecular weight of the component M in the molten steel, unit in kg/mol, M_{M}_{ₓ}_{O}_{_{y}} denotes a molecular weight of the component MₓO_{y} in the inclusions, unit in kg/mol, [MₓO_{y}] denotes a mass fraction of the component MₓO_{y} in the molten steel, unit in %, and b represents the molten steel body;
a sum of the mass transfer flux of the component M in the steel and the mass transfer flux of the component MₓO_{y} in the inclusions is conserved, as shown in an Equation (10):
$${J}_{M} + {J}_{( {M}_{x} {O}_{y} )} = 0 \qquad ( 10 )$$
a total flux of cations and a total flux of anions are equal to maintain an electrically neutral environment, as shown in an Equation (11);
$${\sum }_{M} {n}_{M} {J}_{M} - 2 {J}_{o} - 2 {J}_{s} = 0 \qquad ( 11 )$$
wherein n_{M} denotes a charge number of M, J_{O} denotes a mass transfer flux of the element of O in the molten steel, unit in mol/(m²·s), and J_{S} denotes a mass transfer flux of the element of S in the molten steel, unit in mol/(m²·s);
a concentration of each component within the interface is obtained based on the Equation (2), the Equation (10), and the Equation (11);
a change in the composition of the inclusions over a time step is generated based on an Equation (12):
$$\frac{d ( [ {M}_{x} {O}_{y} ] )}{dt} = - \frac{{A}_{int} {k}_{{M}_{x} {O}_{y}}}{{V}_{inc}} ( [ {M}_{x} {O}_{y} ] * - {[ {M}_{x} {O}_{y} ]}^{b} ) \qquad ( 12 )$$
wherein Aᵢₙₜ denotes an area of the interface, unit in m², V_{inc} denotes a volume of the inclusions, unit in m³, k_{M}_{ₓ}_{O}_{_{y}} is 0.1 times of k_{M}, and k_{M} is determined based on Equations (13) and (14):
$${k}_{M} = 0.2 {( \frac{{D}_{M} \cdot {u}_{slip}}{π {d}_{inc}} )}^{1 / 2} \qquad ( 13 )$$
$${u}_{sinp} = \frac{( {ρ}_{m} - {ρ}_{inc} ) {d}_{inc}^{2} g}{18 {μ}_{st}} \qquad ( 14 )$$
wherein uₛₗᵢₚ denotes a relative diffusion velocity between the molten steel and the inclusions, unit in m/s, d_{inc} denotes the size of the inclusions, in m, g denotes an acceleration of gravity, unit in m/s², μₛₜ denotes a viscosity of the molten steel, unit in Pa·s, and D_{M} denotes a diffusion rate of the component M in the molten steel, unit in m/s.

2. The method for processing entrained slag inclusions in steel with deoxidized calcium with fixed aluminum of claim 1, wherein in response to a CaO index being in a range of 0.52-1, an Al₂O₃ index being in a range of 0.56-2.37, an MgO index being in a range of 0.15-1, and a La₂O₃ index being in a range of 0.29-1, the inclusions are determined as the entrained slag inclusions.

3. The method for processing entrained slag inclusions in steel with deoxidized calcium with fixed aluminum of claim 1, wherein the method further comprises:
generating a calculated composition of the inclusions based on a change in the composition of the inclusions over a plurality of consecutive time steps, the calculated composition characterizing analyzed compositional contents of different components in the inclusions; and storing the calculated composition into an analytical data storage of an analytical terminal.

4. The method for processing entrained slag inclusions in steel with deoxidized calcium with fixed aluminum of claim 1, wherein the method further comprises:
determining a measured composition of the inclusions through sampling and analysis based on an automatic sampling device of a refining furnace, the measured composition characterizing measured compositional contents of different components in the inclusions; and storing the measured composition into an analytical data storage of an analytical terminal.

5. The method for processing entrained slag inclusions in steel with deoxidized calcium with fixed aluminum of claim 4, wherein the method further comprises:
generating a compositional distribution diagram of the inclusions based on the measured composition and a calculated composition;
generating inclusion indices based on the compositional distribution diagram, the inclusion indices including a CaO index, an Al₂O₃ index, a MgO index, and a La₂O₃ index;
determining, based on the inclusion indices, whether the inclusions are the entrained slag inclusions; and in response to the inclusions being the entrained slag inclusions, adjusting a removal frequency of an entrained slag removal device within the refining furnace, and removing the entrained slag inclusions based on the entrained slag removal device.
